(19) United States
(12) Patent Application Publication
(10) Pub. No.: US 2004/0161737 A1
(43) Pub. Date: Aug. 19, 2004

(54) Novel luminescent metal chelates and methods for their detection

(76) Inventors: Dan-Hui D. Yang, Sunnyvale, CA (US); Ganapati R. Mauze, Sunnyvale, CA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/366,885

(22) Filed: Feb. 14, 2003

(51) Int. Cl. C12Q 1/70

(52) U.S. Cl. 435/5

(57) ABSTRACT

The invention provides electrochemiluminescent metal chelates and methods for using these chelates in chemical and biological assays, particularly immunoassays and microarray assays.

### TECHNICAL FIELD OF THE INVENTION

The invention relates to the field of electrochemiluminescent metal chelates for use in the detection of the presence of chemical and biomolecular substances of interest in chemical and biological assays. In particular the invention relates to electrochemiluminescent measurements and techniques that are particularly applicable to immunoassays and microarray technologies.

### BACKGROUND OF THE INVENTION

There is an essential need for rapid and highly specific methods for detecting and quantifying chemical, biological and biochemical substances given the increasing pressures applied to biotechnology and pharmaceutical companies to discover, qualify, and commercially develop new therapeutics. In particular, in this context, there is an ongoing need for new compounds and methods that are capable of detecting small quantities of chemical and biomolecular substances in a fast, cost-efficient manner that can be adapted to high-throughput analytical techniques. A number of existing techniques address the need to detect various biological materials and their quantities. For instance, methods and techniques have been designed for measuring antigen-antibody reactions, protein-ligand system interactions, nucleic acid hybridization, and many polymer-based reactions.

Currently, these systems all operate in a similar fashion based on the presence of a defined label or tag. The label acts as an indicator for the presence or absence of a particular compound, antibody, antigen or other substance of interest. Although these techniques have the advantage of having strong specificity, many suffer from limitations due to cross-reactivity or complex reactions that provide unclear results or even false detections. Often times the actual labels that are used lack the appropriate versatility for use in a variety of assays and techniques simultaneously, something that is crucial for high-throughput assays. In addition, appropriate labels for detection are difficult to find. Often times the labeling method chosen will actually dictate the particular system which needs to be used for detecting an substance of interest. Therefore, preferred labels should be inexpensive, versatile, safe, capable of easy attachment, stable and easily detectable in a variety of solvents. In addition, the labels should not be easily affected by surrounding environment conditions.

More recent assay systems have focused on techniques that use luminescence. These assays eliminate much of the toxic and environmentally dangerous materials, such as radioactive labels. These assays often use very small amounts of materials, thus making them less expensive and resulting in much lower material disposal costs. Among the useful non-radioactive labeling techniques include those employing of organometallic compounds. These compounds are particularly useful because of their versatility as well as their relative rarity of existence in biological systems, thus greatly reducing background noise during signal detection. However, a number of these systems suffer from the lack of sensitivity and can be seldom adapted to more useful systems.

Nevertheless, labels have been made to luminesce through photochemical, electrochemical and chemiluminescent means. Fluorescence and phosphorescence are type of photoluminescence. The former is associated with excitation of compounds with electrons to a singlet state. The latter entails electron transitions from the triplet state to the ground state. The chemiluminescent process, therefore, entails the creation of a luminescent species through the transfer of chemical energy. Electrochemiluminescence entails the creation of a similar luminescent species electrochemically.

A number of ruthenium and osmium complexes are already known and described in the literature and have been used commercially. For instance, a series of complexes for chemiluminescent detection have been disclosed and described in U.S. Pat. No. 5,714,089, U.S. Pat. No. 5,686,244, U.S. Pat. No. 6,048,687, and U.S. Pat. No. 6,140,138. In particular, the complexes disclosed in U.S. Pat. No. 5,714,089 and U.S. Pat. No. 6,140,138 are organometallic compounds containing ruthenium and osmium that are based on the basic bipyridine or phenanthroline structure with at least one functional group binding with chemicals and biomolecules of interest. While an improvement in the art, these compounds suffer from the limitation that they have higher oxidation and redox potentials than would be desirable.

It would, therefore, be desirable to develop novel compounds that have improved oxidation and lower redox potentials. These complexes would provide improved chemiluminescent measurements by lowering overall oxidation and redox potentials. The organometallic compounds described in this invention will provide an improved profile for labeling chemicals and biomolecules and measuring and predicting the consequent biological and physiological effects of these substances.

Thus, it is an object of this invention to provide novel organometallic compounds that can be used in chemiluminescent assays to detect biological and chemical compounds. It is a further object of this invention to provide novel organometallic compounds that can be used in chemiluminescent assays that are performed at lower oxidation and redox potentials that will improve measurement capabilities. Yet another object of this invention is to provide inexpensive, non-radioactive methods of determining the presence of chemicals and biomolecules. Still another object of this invention is to perform such chemiluminescent assays in a cost-efficient manner with good detection sensitivity using these new chemiluminescent compounds.

### SUMMARY OF THE INVENTION

The present invention pertains to the development of a new series of organic and organometallic compounds for chemiluminescence measurements. More specifically, the invention provides organic or organometallic compounds for labeling chemicals and biomolecules of interest and methods for chemiluminescent measurement and detection by imposing electronic fields in sample material in which substances of interest are contained. According to the present invention, there is provided a chemical compound having one of the following formulas:

MLP₂(Bᵤ), ML₂P(Bᵤ)

wherein M is ruthenium or osmium, P is a polydentate ligand of M that can be a substituted or non-substituted bipyridine and/or a substituted or non-substituted phenanthroline, and L is a ligand of M of the following formula:

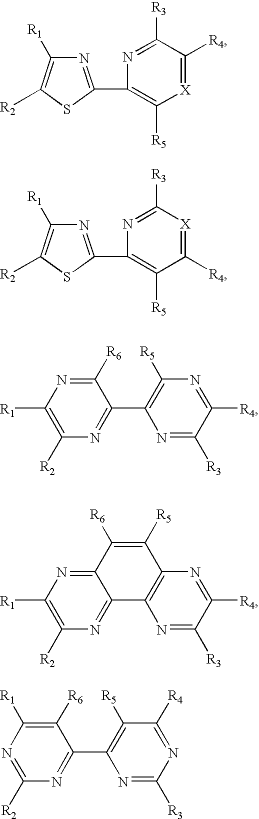

wherein R₁, R₂, R₃, R₄, and R₅ could be H, alkyl, aryl, which can contain amino (NH₂), thiol (SH) and/or carboxyl (COOH) groups for binding with chemicals and biomolecules of interest and X is carbon or nitrogen. Bᵤ is a substance that is attached to the complex through ligand P and/or L and can be a protein, antibody or other biological or chemical material.

The present invention also provides for a compound having the formula:

ML₃(Bᵤ)

where M is ruthenium or osmium and L is a polydentate ligand of M of the following formula:

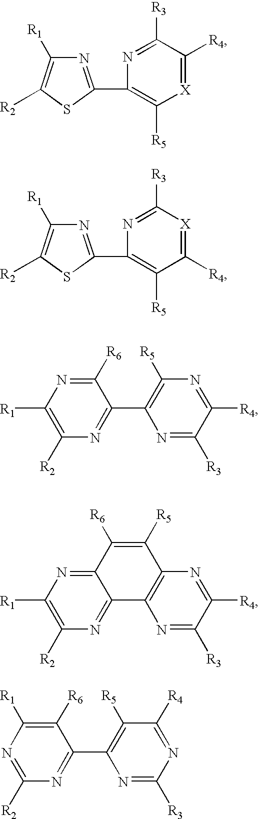

wherein R₁, R₂, R₃, R₄, and R₅ could be H, alkyl, aryl, with at least one amino (NH₂), thiol (SH) and/or carboxyl (COOH) for binding with chemicals and biomolecules of interest and X is carbon or nitrogen. Bᵤ is a substance that is attached to the complex through ligand L and can be a protein, antibody or other biological or chemical material.

The method of the present invention comprises linking Bᵤ with the inventive compound, inducing the compound to emit electromagnetic radiation by exposing the reagent mixture to electrochemical energy; and detecting the electromagnetic radiation that is induced and thereby determining the presence of the compound. The invention further provides for the use and application of the invention in binding methods for determining the presence of a substance of interest. The methods may be used for determining a variety of known or unknown compounds or their mimics, fragments, and combinations in various forms. In addition, the methods may be used to determine labeled substances of interest, to employ labeled substances to determine substances of interest, and/or to use labeled analogues of substances of interest. Furthermore, the invention may be used in competitive binding experiments or in homogenous or heterogeneous binding experiments. In addition, the reactions occurring during an assay protocol can be done either in solution or in solid phase (e.g. microarrays), or in any combination thereof.

### DETAILED DESCRIPTION OF THE INVENTION

Prior to describing the present invention in detail, it is to be understood that this invention includes the variations and derivatives of organometallic compounds that are disclosed. Additionally, the methods disclosed are not limited to specific instrumentation or equipment and it is expressly contemplated that the latter can vary. It is also noted that as used in this specification and the appended claims, the singular forms are expressly meant to include plural forms unless the context clearly dictates otherwise.

According to the present invention, there is provided a chemical compound having one of the following formulas:

MLP₂(Bᵤ), ML₂P(Bᵤ)

where M is ruthenium or osmium, P is a polydentate ligand of M that can be a substituted or non-substituted bipyridine and/or a substituted or non-substituted phenanthroline, and L is a ligand of M of the following formula:

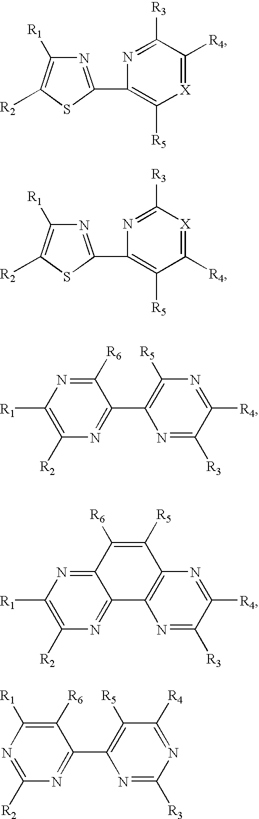

In one embodiment of the invention, M is ruthenium. In another embodiment of the invention M is osmium. The compound has either one or two polydentate ligands P of M. Ligands are compounds that have chemical structures that allow them to specifically bind via covalent, electrostatic, ionic, dipolar, and any other chemical associative mechanism to other defined compounds and/or elements. Polydentate ligands are ligands that can simultaneously bind to several other defined compounds and/or elements. Their chemical structure can either be the same or different while binding to M.

L is also a ligand of M. L has the chemical structure indicated above in which wherein R₁, R₂, R₃, R₄, and R₅ could be H, alkyl, and/or aryl groups. Thus, the R groups can include both aliphatic and aromatic groups as well as mixed aliphatic and aromatic groups that may contain substituents that include amino (NH₂), thiol (SH) and/or carboxyl (COOH) groups. These amino (NH₂), thiol (SH) and/or carboxyl (COOH) groups can occur individually or in combinations or not be present at all. These latter groups are for binding with chemicals and biomolecules of interest. In this context, binding can be any form of chemical interaction, including covalent, electrostatic, ionic, dipolar, and any other associative mechanism of attachment that allow them to specifically bind to other defined compounds and/or elements.

In addition, X is either carbon or nitrogen thus making the ligands heterocyclic as well. Suitable ligands may be unsubstituted, or substituted by any of a large number of substituents known in the art. Suitable substituents include, for example, alkyl, substituted alkyl, aryl, substituted aryl, aralkyl, substituted aralkyl, carboxylate, aldehyde, amide, cyano, amino, hydroxy, imino, hydroxy imino, carbonyl, amidine, guanidiunium, maleimide, sulfur-containing groups, and phosphorus-containing groups.

Bᵤ is a substance of interest that is attached to the complex through ligand P and/or L and can be a protein, antibody or other biological or chemical material. In addition to chemical substances generally, Bᵤ can include many biological and cellular substances including, but not limited to, cells, viruses, subcellullar particles, receptors, proteins, lipoproteins, glycoproteins, peptides, nucleic acids, polysaccharides, lipopolysaccharides, lipids, fatty acids, cellular metabolites, hormones, pharmacological agents, tranquilizers, barbiturates, alkaloids, steroids, vitamins, amino acids, sugars. Other pathogens include, but are not limited to, fungi and nematodes and other organelles or membranes. Also within the scope of the invention are subcellular particles, membrane particles, disrupted cells, fragments of cells and cell walls, ribosomes, multienzyme complexes and other organisms and organism materials that can be derived from living and dead matter. Nucleic acids can include deoxyribonucleic acids (DNAs), tRNA, ribosomal RNA, messenger RNA and other RNAs. Polypeptides and peptides include, for example, enzymes, transport proteins, receptors proteins, structural proteins such as vital coat proteins. Hormones include, and are not limited to, examples such as insulin, thyroid hormone, cardiac glycosides and other related agents. It is also within the scope of the invention to include labeled non-biological substances, including polymeric materials. These substances may be in the form of soluble polymeric molecules, or any of the large variety of known macroscopic forms.

In addition, Bᵤ can be any type of fragment or derivative of the above materials. Bᵤ can also be combinations of the above materials in their entirety or combinations in which fragments and derivatives of more than one material are combined. Additionally, Bᵤ can also be a combination of materials in which some are present in their entirety and others are present in fragmented and/or derivative forms. Also, any suitable mimics of these materials are also appropriate. These could include, for example, but are not limited to, nucleoside, nucleotide, and amino acid analogues or other chemical structures that mimic the conformational and three-dimensional structures of these materials. Mimics can also include functional mimics that cause or imitate the biologically significant effects of the substances of which they are mimics.

It is within the scope of the invention for Bᵤ to be labeled by greater than one, e.g., two, three, four or more, electrochemiluminescent centers and for Bᵤ to be labeled by other suitable materials, including for example, but not limited to, chemical isotopes, both radioactive and non-radioactive. Additionally, Bᵤ can further bind to other chemical and biomolecular substances. This binding can occur via any suitable chemical associative mechanism including covalent, electrostatic, ionic, dipolar, and any other associative mechanism of attachment. Also, the binding to Bᵤ can occur before a reaction with yet another chemical or biomolecular substance, such as, for example, when the organometallic compound contains a ligand that binds to a protein. However, it can also occur as a direct interaction with a substance to be measured during the occurrence of the assay reaction. And it can occur in any intermediate step in a given assay protocol. In addition, the reactions occurring during an assay protocol can be done either in solution or in solid phase (e.g. microarrays), or in any combination thereof. In this context, microarrays are defined as arrays of one- and/or two-dimensional arrangements of addressable regions having particular compounds (usually biopolymers, often nucleotide sequences) associated with that region in which addressable means the microarray has multiple regions of different compounds such that a region at a predetermined location (an address) on the microarray will detect a particular biological or chemical compound or class of compounds bound to a metal chelate of this invention.

In another embodiment of the invention is disclosed a compound having the formula:

ML₃(Bᵤ)

where M is ruthenium or osmium and L is a polydentate ligand of M of the following formula:

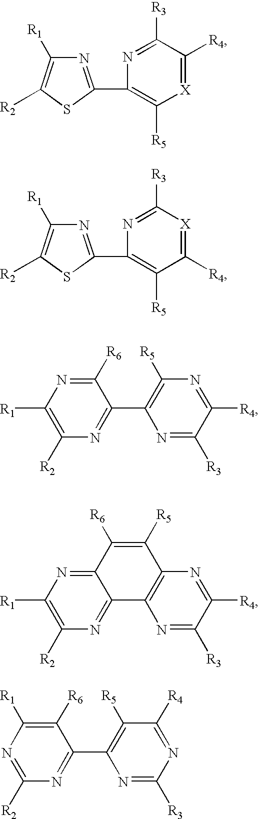

In one embodiment of the invention, M is ruthenium. In another embodiment of the invention M is osmium. The compound has three polydentate ligands L with the chemical structure indicated above wherein R₁, R₂, R₃, R₄, and R₅ could be H, alkyl, and/or aryl groups. Thus, the R groups can include both aliphatic and aromatic groups as well as mixed aliphatic and aromatic groups. At least one of the R groups contains substituents including at least one amino (NH₂), thiol (SH) and/or carboxyl (COOH) group. These amino (NH₂), thiol (SH) and/or carboxyl (COOH) groups can occur individually or in combinations. These latter groups are for binding with chemicals and biomolecules of interest. In this context, binding can be any form of chemical interaction, including covalent, electrostatic, ionic, dipolar, and any other associative mechanism of attachment that allow them to specifically bind to other defined compounds.

In addition, X is either carbon or nitrogen thus making the ligands heterocyclic as well. Suitable ligands may be unsubstituted, or substituted by any of a large number of substituents known in the art. Suitable substituents include, for example, alkyl, substituted alkyl, aryl, substituted aryl, aralkyl, substituted aralkyl, carboxylate, aldehyde, amide, cyano, amino, hydroxy, imino, hydroxy imino, carbonyl, amidine, guanidiunium, maleimide, sulfur-containing groups, and phosphorus-containing groups.

Ligands are compounds that have chemical structures that allow them to specifically bind via covalent, electrostatic, ionic, dipolar, and any other chemical associative mechanism to other defined compounds and/or elements. Polydentate ligands are ligands that can simultaneously bind to several other defined compounds and/or elements whose chemical structure can either be the same or different while binding to M.

Bᵤ is a substance of interest that is attached to the complex, such as a protein, antibody or other biological or chemical material. In addition to chemical substances generally, Bᵤ can include many biological and cellular substances including, but not limited to, cells, viruses, subcellullar particles, receptors, proteins, lipoproteins, glycoproteins, peptides, nucleic acids, polysaccharides, lipopolysaccharides, lipids, fatty acids, cellular metabolites, hormones, pharmacological agents, tranquilizers, barbiturates, alkaloids, steroids, vitamins, amino acids, sugars. Other pathogens include, but are not limited to, fungi and nematodes and other organelles or membranes. Also within the scope of the invention are subcellular particles, membrane particles, disrupted cells, fragments of cells and cell walls, ribosomes, multienzyme complexes and other organisms and organism materials that can be derived from living and dead matter. Nucleic acids can include deoxyribonucleic acids (DNAs), tRNA, ribosomal RNA, messenger RNA, and other RNAs. Polypeptides and peptides include, for example, enzymes, transport proteins, receptors proteins, structural proteins such as vital coat proteins. Hormones include, and are not limited to, examples such as insulin, thyroid hormone, cardiac glycosides and other related agents. It is also with in the scope of the invention to include labeled non-biological substances, including polymeric materials. These substances may be in the form of soluble polymeric molecules, or any of the large variety of known macroscopic forms.

In addition, Bᵤ can be any type of fragment or derivative of the above materials. Bᵤ can also be combinations of the above materials in their entirety or combinations in which fragments and derivatives of more than one material are combined. Additionally, Bᵤ can also be a combination of materials in which some are present in their entirety and others are present in fragmented and/or derivative forms. Also, any suitable mimics of these materials are also appropriate. These could include, for example, but are not limited to, nucleoside, nucleotide, and amino acid analogues or other chemical structures that mimic the conformational and three-dimensional structures of these materials. Mimics can also include functional mimics that cause or imitate the biologically significant effects of the substances of which they are mimics.

It is within the scope of the invention for Bᵤ to be labeled by greater than one, e.g., two, three, four or more, electrochemiluminescent centers and for Bᵤ to be labeled by other suitable materials, including for example, but not limited to, chemical isotopes, both radioactive and non-radioactive. Additionally, Bᵤ can further bind to other chemical and biomolecular substances. This binding can occur via any suitable chemical associative mechanism including covalent, electrostatic, ionic, dipolar, and any other associative mechanism of attachment. Also, the binding to Bᵤ can occur before a reaction with yet another chemical or biomolecular substance, such as, for example, when the organometallic compound contains a ligand that binds to a protein. However, it can also occur as a direct interaction with a substance to be measured during the occurrence of the assay reaction. And it can occur in any intermediate step in a given assay protocol. In addition, the reactions occurring during an assay protocol can be done either in solution or in solid phase (e.g. microarrays), or in any combination thereof. In this context, microarrays are defined as arrays of one- and/or two-dimensional arrangements of addressable regions having particular compounds (usually biopolymers, often nucleotide sequences) associated with that region in which addressable means the microarray has multiple regions of different compounds such that a region at a predetermined location (an address) on the microarray will detect a particular biological or chemical compound or class of compounds bound to a metal chelate of this invention.

The invention is illustrated in the examples that follow. These examples are set forth to aid in understanding of the invention but are not intended to, and should not be construed to, limit in any way the invention as set forth in the claims which follow thereafter.

### EXAMPLE 1

### Preparation of Ruthenium bis (tap(1,4,5,8-tetraazaphenanthrene))

Ruthenium trichloride (0.15 nmole) and lithium chloride (0.1 mmole) are dissolved or suspended in about 20 mL DMF (N,N-dimethylformamide). Tap (0.30 mmole) is then added. The reaction mixture is refluxed overnight. After cooling in an ice bath, 50 mL of ice water is added. The precipitated solid that appears is filtered under vacuum. The recovered solid is washed thoroughly with water until colorless.

### EXAMPLE 2

### Preparation of Ruthenium bis (tap) (2,2′-bipyridine-4,4′-dicarboxylic acid)

The ruthenium bis (tap) chloride salt synthesized in Example 1 above is dissolved or suspended in ethylene glycol. 2,2′-bipyridine-4,4′-dicarboxylic acid is then added (1:1 mole ratio with ruthenium bis (tap)). The mixture is refluxed under argon atmosphere for 30 to 60 minutes until it turns a bright orange color. Most of the ethylene glycol is evaporated under heating and argon gas flow. 50 mL of ice water is then added, followed by 30 mL of a saturated solution of ammonium hexafluorophosphate. The precipitated product is filtered under vacuum and then purified by chromatography. The product is dried in a vacuum dessicator. If it is difficult to form a precipitate, the pH of the solution can be adjusted to about 3.

### EXAMPLE 3

### Preparation of the Activated Ester of Ruthenium bis (tap) (2,2′-bipyridine-4,4′-dicarboxylic acid

Ruthenium bis (tap) (2,2′-bipyridine-4,4′-dicarboxylic acid) is dissolved in anhydrous DMF. Dicyclohexylcarbodiimide and N-hydroxylsuccinimide are then added (the ratio of the three reagents should be about 1:2.2:2.2). The mixture is stirred at room temperature for about 5 hours. The precipitated solid is filtered out. The collected solution contains activated ruthenium complex.

### EXAMPLE 4

### Labeling of Proteins with Activated Ruthenium Complexes Using Human Serum Albumin (HSA) as an Example

HSA is dissolved in a 50 mM carbonate buffer (pH 8.4-9.5). The above activated ruthenium complex in DMF is added to the protein solution under constant stirring (the ratio of protein to ruthenium complex typically ranges from 10 to 50 depending on the degree of labeling desired). The mixture is stirred at room temperature for about 3-4 hours or at 4 degrees Centigrade overnight. The ruthenium complex-conjugated protein is purified by using G-50 chromatography using a 10 mM phosphate buffered saline (PBS) buffer as an eluent or by performing extensive dialysis using 10 mM PBS buffer. The fastest moving orange band on the G-50 chromatographic column is the protein conjugate.

### EXAMPLE 5

### Labeling of Nucleic Acids with Activated Ruthenium Complexes

A terminally modified nucleic acid with a thiol functional group will be created on one end of the nucleic acid molecule while the other end will contain a primary amine functional group. The primary amine group can be reacted with the activated ruthenium complex from Example 3 above in a carbonate buffer. The conjugated product can be purified by high performance liquid chromatography (HPLC) or preparative thin layer chromatography (TLC). The substances produced can be stored at temperatures below the freezing point.

### EXAMPLE 6

### Attachment of Probes to Substrates

Labeled substances in Example 5 can also be deposited on a solid phase through the thiol modification at the other end of the molecule. The solid substrate can be any suitable material, including the electrodes themselves whose surfaces can be modified with reactive groups as indicated below in Example 7. For example, thiol-terminated terminated nucleic acids to which the electrochemiluminescent species has been attached can be reacted with a gold or nickel surface, including that of an electrode. Thiol can also react with other functionalized surfaces such as a maleimide-modified surface.

### EXAMPLE 7

### Electrochemiluminescent Detection of Ruthenium Labeled Samples

Electrochemiluminescent measurements can be carried out in a variety of detection devices, including, for example, a one-compartment cell with an optically flat bottom. The working electrode can be glassy carbon, gold, nickel, or a similar type of material, and the counter electrode can be platinum or a similar type material. Also, electrodes may have surface modifications that include carboxyl, amino, thiol, and hydroxyl groups that are capable of reacting with the organometallic compounds. A reference electrode also needs to be incorporated into such a device. Light intensity measurements will be made once the organometallic complex is induced to electrochemiluminesce by applying a potential to the electrodes. Detection will be accomplished using a photomultiplier tube and integrating the resulting signals with a recorder or similar type instrument. The elctrochemiluminescent-labeled materials can be in solution. For example, such material may be detected upon binding with detection probes attached to the electrode surface. The ruthenium-labeled complex can also be attached to a substrate. For example, the thiol-modified nucleic acid molecules described in Example 5 would generate electrochemiluminescent signals upon binding with a nucleic acid molecule of complementary sequence to form a double-stranded structure. Other possible modifications to increase the signal measured could be made to facilitate the electron transfer in the complex, such as linking conducting molecules on the DNA side chains.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A chemical compound having one of the following formulas:
MLP₂(Bᵤ), ML₂P(Bᵤ)
where M is selected from the group consisting of ruthenium and osmium, P is a polydentate ligand of M selected from the group consisting of substituted bipyridines, non-substituted bipyridines, substituted phenathrolines, non-substituted phenanthrolines, and any combinations thereof, and L is a ligand of M wherein L is selected from the following: 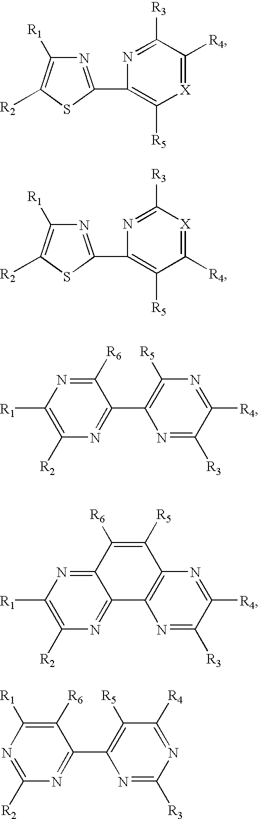
wherein R₁, R₂, R₃, R₄, and R₅ could be H, alkyl, aryl, with any alkyl and aryl group having substituents selected from the moieties consisting of amino (NH₂), thiol (SH), and carboxyl (COOH) groups, either individually or any combination thereof, including multiple or no substituents of each group, for binding with chemicals and biomolecules of interest; X is selected from the group consisting of carbon and nitrogen; and Bᵤ is a substance that is attached to the complex through P, L, or both.

2. A chemical compound having the formula:
ML₃(Bᵤ)
where M is selected from the group consisting of ruthenium and osmium and L is a polydentate ligand of M wherein L is selected from the following: 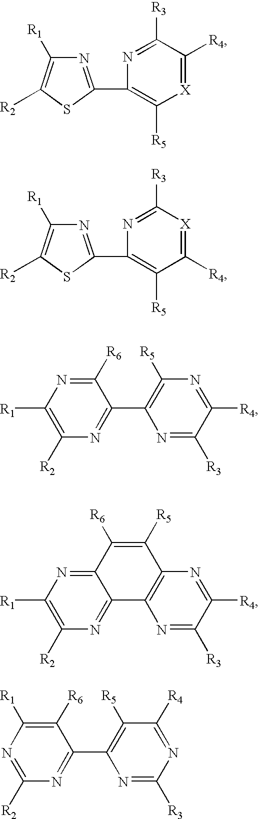
wherein R₁, R₂, R₃, R₄, and R₅ could be H, alkyl, aryl, with any alkyl and aryl group having substituents selected from the moieties consisting of amino (NH₂), thiol (SH), and carboxyl (COOH) groups, either individually or any combination thereof, including multiple substituents of each group wherein at least one of said substituents is present, for binding with chemicals and biomolecules of interest; X is selected from the group consisting of carbon and nitrogen; and Bᵤ is a substance that is attached to the complex through L.

3. A method of detecting the presence of a substance of interest in a sample, comprising the steps of:
a) contacting the sample and a chemical compound, the chemical compound having one of the following formulas:
MLP₂(Bᵤ), ML₂P(Bᵤ)
where M is selected from the group consisting of ruthenium and osmium, P is a polydentate ligand of M selected from the group consisting of substituted bipyridines, non-substituted bipyridines, substituted phenathrolines, non-substituted phenanthrolines, and any combinations thereof, and L is a ligand of M wherein L is selected from the following: 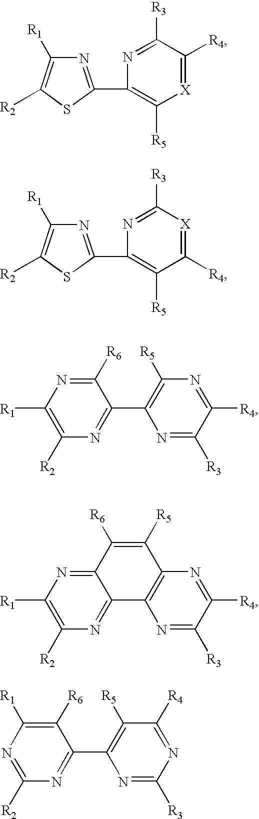
wherein R₁, R₂, R₃, R₄ and R₅ could be H, alkyl, aryl, with any alkyl and aryl group having substituents selected from the moieties consisting of amino (NH₂), thiol (SH), and carboxyl (COOH) groups, either individually or any combination thereof, including multiple or no substituents of each group, for binding with chemicals and biomolecules of interest; and X is carbon or nitrogen and Bᵤ is a substance that is attached the complex through P, L, or both;
b) inducing the chemical compound to electrochemiluminesce by exposing the chemical compound to electrochemical energy; and
c) detecting emitted electrochemiluminescence and thereby detecting the substance of interest.

4. The method of claim 3 further comprising the addition of one or more additional chemical compounds.

5. The method of claim 3 further comprising the step of selecting Bᵤ from the group consisting of whole cells, viruses, subcellular particles, receptors, proteins, antibodies, lipoproteins, glycoproteins, peptides, nucleic acids, polysaccharides, lipopolysaccharides, lipids, fatty acids, cellular metabolites, hormones, pharmacological agents, tranquilizers, barbiturates, alkaloids, steroids, vitamins, amino acids, sugars, mimics of these substances, fragments of these substances, combinations of these substances, combinations of fragments of these substances, and combinations containing these substances and fragments thereof.

6. The method of claim 3 further comprising the step of detecting the presence of a substance of interest and Bᵤ is a compound capable of specifically binding to said substance of interest or Bᵤ can compete with said substance of interest in a specific binding assay.

7. The method of claim 3, wherein Bᵤ is a compound of biological origin or a compound having biological activity.

8. The method of claim 3, wherein Bᵤ is an antibody or fragment thereof.

9. The method of claim 3, wherein Bᵤ is a receptor or fragment thereof.

10. The method claim 3, wherein Bᵤ is a nucleic acid.

11. The method of claim 3, wherein Bᵤ is a substance that can specifically bind with another substance.

12. The method of claim 3, wherein Bᵤ is attached to the complex via covalent bonds.

13. The method of claim 3 further comprising the step of binding Bᵤ to the metal-containing compound prior to conducting an assay.

14. The method of claim 3 further comprising the step of binding Bᵤ to the metal-containing compound during the process of conducting an assay.

15. The method of claim 3 further comprising the step of performing the assay in solution.

16. The method of claim 3 further comprising the step of binding the substance of interest to a surface.

17. The method of claim 16 further comprising the step of including the surface in a microarray assay system.

18. A method of detecting the presence of a substance of interest in a sample, comprising the steps of:
a) contacting the sample and a chemical compound, the chemical compound having the formula:
ML₃(Bᵤ)
where M is selected from the group consisting of ruthenium and osmium and L is a polydentate ligand of M wherein L is selected from the following: 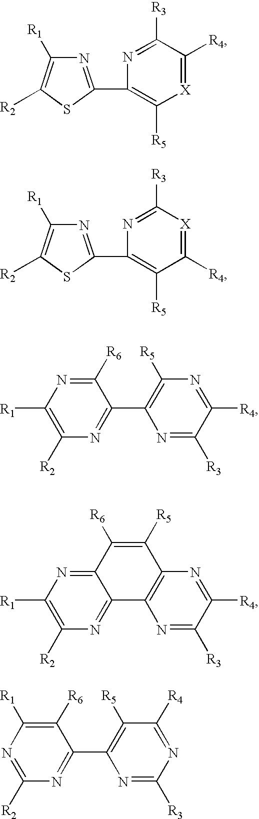
wherein R₁, R₂, R₃, R₄ and R₅ could be H, alkyl, aryl, with any alkyl and aryl group having substituents selected from the moieties consisting of amino (NH₂), thiol (SH), and carboxyl (COOH) groups, either individually or any combination thereof, including multiple substituents of each group wherein at least one of said substituents is present; for binding with chemicals and biomolecules of interest; X is selected from the group consisting of carbon and nitrogen; and Bᵤ is a substance that is attached to the complex through L;
b) inducing the chemical compound to electrochemiluminesce by exposing the chemical compound to electrochemical energy; and
c) detecting emitted electrochemiluminescence and thereby detecting the substance of interest.

19. The method of claim 18 further comprising the step of adding one or more additional chemical compounds.

20. The method of claim 18 further comprising the step of selecting Bᵤ from the group consisting of whole cells, viruses, subcellular particles, receptors, proteins, antibodies, lipoproteins, glycoproteins, peptides, nucleic acids, polysaccharides, lipopolysaccharides, lipids, fatty acids, cellular metabolites, hormones, pharmacological agents, tranquilizers, barbiturates, alkaloids, steroids, vitamins, amino acids, sugars, mimics of these substances, fragments of these substances, combinations of these substances, combinations of fragments of these substances, and combinations containing these substances and fragments thereof.

21. The method of claim 18 further comprising the step of detecting the presence of a substance of interest and Bᵤ is a compound capable of specifically binding to said substance of interest or Bᵤ can compete with said substance of interest in a specific binding assay.

22. The method of claim 18, wherein Bᵤ is a compound of biological origin or a compound having biological activity.

23. The method of claim 18, wherein Bᵤ is an antibody or fragment thereof.

24. The method of claim 18, wherein Bᵤ is a receptor or fragment thereof.

25. The method claim 18, wherein Bᵤ is a nucleic acid.

26. The method of claim 18, wherein Bᵤ is a substance that can specifically bind with another substance.

27. The method of claim 18, wherein Bᵤ is attached to the complex via covalent bonds.

28. The method of claim 18 further comprising the step of binding Bᵤ to the metal-containing compound prior to conducting an assay.

29. The method of claim 18 further comprising the step of binding Bᵤ to the metal-containing compound during the process of conducting an assay.

30. The method of claim 18 further comprising the step of performing the assay in solution.

31. The method of claim 18 further comprising the step of binding the substance of interest to a surface.

32. The method of claim 31 further comprising the step of including the surface in a microarray assay system.
